(19) United States
(12) Patent Application Publication
(10) Pub. No.: US 2003/0158585 A1
(43) Pub. Date: Aug. 21, 2003

(54) Method and apparatus for electromagnetic stimulation of nerve, muscle, and body tissues

(76) Inventor: Daniel R. Burnett, Menlo Park, CA (US)

Correspondence Address: MAINE & ASMUS, 100 MAIN STREET, P O BOX 3445, NASHUA, NH, 03061-3445, US

(21) Appl. No.: 10/266,535

(22) Filed: Oct. 8, 2002

Related U.S. Application Data
(63) Continuation-in-part of application No. 10/077,434, filed on Feb. 19, 2002.
(60) Provisional application No. 60/380,132, filed on May 6, 2002.

(51) Int. Cl. A61N 1/00

(52) U.S. Cl. 607/2; 600/13

(57) ABSTRACT

An electromagnetic stimulating system and components configured to provide stimulation to tissues of the human body, including nerves, muscles (including superficial and deep muscles), or other body tissues without significant discomfort to the patient. The system utilizes an ergonomic, body-contoured, and conformable appliance to encase a transducer intended to deliver pulses of electromagnetic stimulation to targeted regions of the body. Transducer configurations include a substantially flat coil, a circumferential uninterrupted solenoid, and a circumferential, substantially solenoidal structure having an openable joint formed by a multiple conductor connector buckle. Index markings appliance allow for repetitive application, for more consistent therapy targeting specific anatomic regions with therapeutic pulsed electromagnetic fields.

### CROSS REFERENCE TO RELATED APPLICATIONS

This patent application is related to and claims priority of U.S. Provisional Patent Application No. 60/380,132, filed May 6, 2002, entitled: METHOD AND APPARATUS FOR ELECTROMAGNETIC STIMULATION OF NERVE, MUSCLE, AND BODY TISSUES, which is hereby incorporated by reference for all purposes; this application is also a continuation in part of, is related to, and claims priority of co-pending U.S. Non-Provisional application Ser. No. 10/077,434, filed Feb. 19, 2002, entitled: METHOD AND APPARATUS FOR ELECTROMAGNETIC STIMULATION OF NERVE, MUSCLE, AND BODY TISSUES, which is hereby incorporated by reference for all purposes.

### FIELD OF THE INVENTION

The present invention relates to the field of medical devices, in particular electromagnetic stimulating devices for stimulation of nerve, muscle, and/or other body tissues with applications in the field of medicine.

### BACKGROUND

The concept of pulsed electromagnetic stimulation (PES) was first observed by the renowned scientist Michael Faraday in 1831. Faraday was able to demonstrate that time varying, or pulsed electromagnetic fields have the potential to induce current in a conductive object. Faraday's experimental setup was simple. He found that by passing strong electric current through a coil of wire he was able to produce pulsed electromagnetic stimuli. This pulsed electromagnetic stimulus was able to induce the flow of current in a nearby electrically conductive body.

In the years since the discoveries of Faraday, pulsed electromagnetic stimulators have found application in countless areas of scientific investigation. In 1965, the scientists Bickford and Freming demonstrated the use of electromagnetic stimulation to induce conduction within nerves of the face. Later, in 1982 Poison et al., U.S. Pat. No. 5,766,124 produced a device capable of stimulating peripheral nerves of the body. This device was able to stimulate peripheral nerves of the body sufficiently to cause muscle activity, recording the first evoked potentials from electromagnetic stimulation. One of the earliest practical applications of electromagnetic stimulating technology took the form of a bone growth stimulator a device that employed low frequency pulsed electromagnetic fields (PEMF) to stimulate bone repair. They first found use approximately 20 years ago in the treatment of non-healing fractures, and are slowly becoming the standard of care for this condition.

As investigators have studied the effects of electromagnetic fields on fracture healing, it has been demonstrated that PEMFs can not only facilitate fracture healing but also promote numerous other positive effects on the human body, including: (1) causing muscles to contract, (2) altering nerve signal transmission to decrease experienced pain, and (3) causing new cell growth in cartilage. These powerful effects of pulsed electromagnetic stimulation have been well-established in laboratory studies of animal models and also in multiple large, double-blind, placebo-controlled studies of human subjects published in the medical literature.

Existing pulsed electromagnetic stimulation devices have taken a number of different forms in attempts to treat various medical conditions. These different forms have resulted in two broad categories of coil arrangements for the generation of PEMFs: (1) planar or semi-planar designs with tightly wound coils, and (2) solenoid coils. Flat, wound coils create electromagnetic fields that degrade rapidly over a short distance as they pulse away from the inducing coil.

Solenoid type coils create pulsed electromagnetic fields inside the coil that are relatively uniform throughout, with a peak field strength at the center of the coil. Examples of existing devices with tightly wound coil arrangements include:

Erickson's U.S. Pat. No. 5,181,902, Jan. 26, 1993, which describes a device using a double transducer system with contoured, flat wound transducers intended to generate therapeutic flux-aided electromagnetic fields in the body. The device is suggested to be conformed to the contour of the patient's back and incorporates an adjustable belt into the design. This system, as it is described, is disadvantageous in at least two respects. First, the flat, wound nature of the coil in this device is limited in its delivery of pulsed electromagnetic fields to deep tissues of the body. Second, the rigid nature of this device, intended to provide bracing for patients recovering from spinal fusion surgeries, may prove uncomfortable to some patients, especially in delivering therapy to regions of the body other than the back, such as the knee, elbow, hand, or other joints and tissues.

U.S. Pat. No. 6,086,525, which discloses a device that has a single coil in the shape of a “C” where the intensity of the electromagnetic field is between the ends of the “C”. That point must be employed directly over the target nerve or muscle to be stimulated. The coil is toroidal in configuration and utilizes a unique core of vanadium permendur in the preferred form. One of the disadvantages of this device is that it requires a trained technician to treat the patient and to properly hand hold the open end of the “C” over the targeted nerve or muscle to be stimulated. The device is not portable and is designed for use in hospitals or similar institutions. Also the vanadium permendur core is required to increase the strength of the electromagnetic field to be strong enough to be effectively used. The design, shape and configuration described in Davey and other prior art devices, require the electromagnetic stimulator to be hand operated during use.

Tepper in U.S. Pat. No. 5,314,401, May 24, 1994 describes a pulsed electromagnetic field transducer that is intended to be conformable to the contour of a patients body. The PEMF transducer in this application as having a desired form and sufficient rigidity to maintain an anatomical contour. This system is disadvantageous in a number of respects. First, the desired contouring of this device will require that a significant number of different sizes be manufactured to accommodate the contours of an endless variety of body shapes. Second, the intended device does not incorporate markings to ensure that the device is placed in a correct alignment over the targeted area of the body. Finally, this proposed device utilizes flat, wound coils, providing PEMFs that do not penetrate as deeply or as uniformly into body tissues as those fields produced by solenoid coils.

In U.S. Pat. No. 6,179,770 B1, Jan. 30, 2001, Mould describes dual coil assemblies in a magnetic stimulator for neuro-muscular tissue, with cooling provided for the transducer coil. This device is intended to be held by a trained user over the targeted regions of the body in order to deliver PEMF therapy. The design of this device is limited by the difficult nature of manipulating a single coil and the cost-intensive requirement of using highly skilled medical personnel for operation.

Parker in U.S. Pat. No. 6,155,966, Dec. 5, 2000 describes a wearable article with a permanent magnet/electromagnet combination device to be used for toning tissue with focused, coherent EMF. This device is disadvantageous in several respects. First, this device is intended to be a hand-held application, with the user applying the device to targeted areas of the body. The hand-held nature of this application creates an inherently inconsistent and non-uniform method for delivery, especially difficult with the intention of the device to provide a focused electromagnetic stimulus. Second, the device combines a static magnet with the electromagnet assembly in an attempt to create a unipolar, negative polarity field. This form of electromagnetic field stimulation has not been demonstrated to be effective in the treatment of osteoarthritis, musculoskeletal pain, or atrophy treatment—conditions for which the present invention will provide therapy.

March's U.S. Pat. No. 6,200,259 B1, Mar. 13, 2001 describes a device with electromagnetic field coils applied front and back to a patient for treating cardiovascular disease by angiogenesis. An EMF dosage plan contemplates, multiple coil implants and pulse variables including carrier frequency, pulse shape, duty cycle, and total time exposed. This device describes the placement of coils around the regions of tissues in which collateralization of blood flow (or angiogenesis) is desired. The design contemplates applications including the use of coils embedded in a cloth wrap, which could be worn as a garment surrounding the body area of interest. Alternatively, a wrap with embedded coils to be placed around an arm or a leg to deliver the desired field is described. The use of PEMF in this application for the purpose of modulation of angiogenesis shows significant promise. The description of this device, however, does not suggest any extension of the electromagnetic phenomenon in circumstances where PEMF stimulation can provide dramatic opportunities for the treatment of osteoarthritis, and musculoskeletal pains including tendonitis, bursitis, and muscle spasms. Furthermore, this invention does not provide for the use of solenoid-type coils for the delivery of PEMF.

Polson's U.S. Pat. No. 5,766,124, Jun. 16, 1998 describes a magnetic stimulator of neuro-muscular tissue. The primary aim of this invention is devise a reserve capacitor providing more efficiency in the control circuitry. The description of the device, however, describes the stimulating coil in broad, generic terms, and does not contemplate application of the coil in any type of body wrap or other specific method for delivering PEMF to targeted areas of the body. As a result, this device is disadvantageous, in the respect that is does not provide for any method or delivery system to provide consistent, uniform PEMF stimulation.

Schweighofer's U.S. Pat. No. 6,123,658, Sep. 26,2000 describes a magnetic stimulation device which consists of a stimulation coil, a high-voltage capacitor, and a controllable network part. This device is intended to differentiate itself from low-voltage, low current devices by using a specific high voltage, high current design to deliver PEMF for the purpose of triggering action potentials in deep neuromuscular tissue. This device, however, does not contemplate the incorporation of the stimulation coil into ergonomic body wraps for the purpose of delivering consistent, user-friendly therapy. Instead, the coil is described as having a difficult and expensive to use hand-held configuration.

Lin in U.S. Pat. No. 5,857,957, issued Jan. 12, 1999 teaches the use of functional magnetic stimulation for the purpose of inducing a cough function in a mammalian subject. The description of the device provides for the use of hand-held stimulation coil, intended to be placed over the anterior chest of the subject for the purpose of stimulating nerves to induce a cough. This system is disadvantageous in the requirement of hand-held delivery which is difficult and inconsistent. The description contemplates use of the device in the induction of cough, and does not contemplate extension of the use of the device into other areas of neuromuscular stimulation.

Tepper in U.S. Pat. No. 6,024,691, issued Feb. 15, 2000 describes a cervical collar with integral transducer for PEMF treatment. The description of this device provides for the use of a single coil transducer, formed into the shape of a cervical collar. This system is disadvantageous in several respects. First, this device does not provide for the use of solenoid-type coils in the delivery of PEMF, which can provide a more uniform and consistent signal. Second, the semi- rigid design of the collar complicates the delivery of PEMF to persons of differing body sizes. That is, for a person with a larger than average (or smaller than average) size neck, the design and semi-rigid nature of the device would make an exact fit difficult, thereby diminishing the effectiveness of any delivered therapy. Furthermore, this device is designed to immobilize the neck and is therefore not applicable to most patients. Whereas, with a flexible, ergonomic delivery system for PEMF stimulation, various sizes of wraps can accommodate nearly any type of body habitus. Lastly, the device must be lowered over the head making application difficult versus the invention found in FIGS. 4 and 6 where the coil can be opened to allow entrance of the body part.

Erickson in U.S. Pat. No. 5,401,233, issued Mar. 28, 1995 describes a neck collar device for the delivery of PEMF therapy. The description of this device provides for the use of semi-rigid transducers, intended to be conformable to a selected anatomical contour. This device in disadvantageous in respects similar to those of Pollack U.S. Pat. No. 5,401,233, in that the device does not provide for the use of solenoid-type coils. Furthermore, this device is intended to provide bracing (as might be necessary for the treatment of fractures or after surgery). As a result, the rigidity of the device necessary to serve the bracing function makes the device less comfortable to wear, especially for a person who would not require bracing (such as in the treatment of arthritis, muscle spasm, or other forms of musculoskeletal pain).

While the discussion of prior art above related primarily to devices employing flat, wound coils in the delivery of PEMF, there are a handful of devices that contemplate the use of solenoid-type coils. Examples include:

Kolt in U.S. Pat. No. 5,518,495, issued May 21, 1996 describes a coil wound on a large bobbin that permits the insertion of an arm or a leg into the field of the coil for PEMF type therapy. This device is disadvantageous in several respects. First, the described use of a bobbin, around which the wire for the stimulating coil is wound provides for the treatment of certain areas of the body, but is certainly limited in its ability to deliver therapy to areas of the body such as the hips, shoulder, back, neck, etc. That is, the constraints of human anatomy make it nearly impossible to approximate a metal bobbin, and thus the stimulating coil, to regions of the body such as the ball and socket joints of the hip or shoulder, where the round metal bobbin would strike the torso before it allowed the stimulating coils to adequately blanket with therapy the head of arm or and joint in the hip and shoulder. Similarly, the use of a metal bobbin for the delivery of PEMF stimulation to the back would necessitate a large, cumbersome delivery system (into which the entire body would have to fit) in order to adequately deliver stimulation to targeted areas on the back or torso. An ergonomic body wrap, incorporating a solenoid-type coil would prove much more effective in delivering PEMF stimulation directly to the targeted areas.

Second, the device is described as a rigid bobbin through which the extremity is placed. This format makes application more difficult in that the applicator cannot be worn and therefore does not provide for consistent ideal placement of the extremity to maximize field effects. In fact, most designs of a similar nature are clinic-based devices and, therefore, would not be amenable to home healthcare applications as with the current invention.

Third, the device described magnetic field within the bobbin is intended to have a maximum magnetic flux density in the range of 4.5 to 6 gauss. Studies such as by Trock et al in the Journal of Rheumatology 1994; 21(10): 1903-1911, have shown that PEMF stimulation in the range of 15-25 or more gauss are effective in the treatment of osteoarthritis or other musculoskeletal pain conditions.

Pollack in U.S. Pat. No. 5,014,699, issued May 14, 1991 describes a coil wound around the cast on an appendage for the delivery of PEMF treatment to fractured bone. The described device has shown promise for the treatment of fractured bone, especially nonunion or delayed healing fractures. However, the description of the device does not provide for extension of this application to the treatment of other conditions, such as arthritis, musculoskeletal pain, or atrophy. Moreover, the described device does not provide for the extension of the use of an ergonomic, body contoured wrap in the delivery of PEMF.

Clearly what is needed is to integrate electromagnetic stimulation technology and the delivery of PEMF therapy into a user-friendly, body-contoured applicator. The delivery system of this device should use solenoid-type coils to effectively carpet the entire targeted anatomic regions of the body. Such a device should build on existing electromagnetic stimulator technology, which has in disparate forms attempted to use various different types of stimulating coils and body applications to PEMF therapy. The known technologies, however, have not contemplated the use of a delivery system for PEMF that incorporates the together the elements of: (1) an ergonomic, body contoured wrap that is coded with clear markings to provide for repetitive application and consistent therapy onto the same body area, (2) the use of solenoid-type coils for the delivery of uniform, consistent PEMF stimulation and (3) the use of solenoid-type coils that can be opened and placed over the treatment region making application easier and more effective.

### SUMMARY OF THE INVENTION

The sum of the elements of the present invention have provided for the development and manufacturing of an easy-to-use, ergonomically designed system that will have applications within a host of clinical and home ease of use health applications. The invention simply stated is drawn to an electromagnetic stimulating system able to provide stimulation to tissues of the human body, including nerves and muscles, both superficial and deep muscles, and/or other body tissues without significant discomfort to the patient. This electromagnetic stimulating system utilizes solenoid coils and solenoid coils with a buckle interface in conjunction with a conformal body appliance for the delivery of PEMF stimulation. The coils are encased in an ergonomic, body-contoured appliance that is coded with clear markings to provide for repetitive application and consistent therapy onto the same body area. The design of the appliance is intended to allow for ease of use and also for the targeting of anatomic regions to be exposed to the impulses of the PEMFs.

The several embodiments have been developed and manufactured to provide PEMF stimulation in a format that will thoroughly blanket the designated therapeutic area and to provide consistent therapy that can be quickly and easily administered. The invention is designed to be patient user friendly as well as to be portable. It can be used in a hospital, an outpatient clinic, a therapist's office, or even at a patient's home.

It is an object of the present invention to provide an electromagnetic system and components for stimulating regions of the body, employing solenoid coils and solenoid coils with a buckle interface that can be fired sequentially or in unison depending on the particular required treatment conditions requiring both maximal stimulation sufficient to cause contraction of muscle fibers as well as submaximal stimulation which will be sufficient to provide therapy but not to cause contraction of muscle fibers. The applications of the system can be divided into maximal and submaximal categories, in which the former requires significantly higher levels of inducting current than the latter. The maximal applications of the device include: (1) non-invasive stimulation of the peripheral nervous system; (2) treatment and/or prevention of atrophy as would be therapeutic during recovery after a person sustains a fracture, experiences paralysis of a limb or other body part, or undergoes surgery, such as ACL repair in the knee; and (3) treatment of neurogenic bladder and bowel. Submaximal applications of the device include treatment of musculoskeletal pain (e.g. back and neck pain, muscle spasms, and other forms of muscle or skeletal related pain); and treatment of arthritis. The components may be variously combined for optimal effect.

It is an object of the invention to provide a system for delivering PEMF stimulation to selective anatomic regions of the body, utilizing an ergonomic appliance designed to facilitate accurate and targeted delivery of therapy. The applicator has been developed and manufactured to be coded with clear markings to provide for repetitive application and consistent therapy onto the same body area of the body. This design will facilitate the placement of the device for the stimulation of key nerves, muscles, and/or body tissues. The appliance may be of various forms.

It is another object of the present invention to provide an electromagnetic system and components for stimulating regions of the body, which has solenoid coils and solenoid coils with a buckle interface that can be fired sequentially or in unison depending on the particular required treatment conditions requiring both maximal stimulation sufficient to cause contraction of muscle fibers as well as submaximal stimulation which will be sufficient to provide therapy but not to cause contraction of muscle fibers. The applications of the system can be divided into maximal and submaximal categories, in which the former requires significantly higher levels of inducting current than the latter. The maximal applications of the device include: (1) non-invasive stimulation of the peripheral nervous system; (2) treatment and/or prevention of atrophy as would be therapeutic during recovery after a persons sustains a fracture, experiences paralysis of a limb or other body part, or undergoes surgery, such as ACL repair in the knee; and (3) treatment of neurogenic bladder and bowel. Submaximal applications of the system include: one, treatment of musculoskeletal pain (e.g. back and neck pain, muscle spasms, and other forms of muscle or skeletal related pain); and two, treatment of arthritis.

It is an object of the invention to provide a system to electromagnetically stimulate selective nerves muscles, and/or body tissues including an appliance component that is user friendly and capable of being used even by an unskilled patient in a home healthcare setting to obtain repetitive placement for continuing treatments.

It is a further object of the invention to provide a system and components to electromagnetically stimulate selective nerves, muscles, and body tissues to provide consistent therapy, with the ergonomic wrap appliance targeting key nerves and eliminating the requirement for a highly trained operator to manipulate the device.

Still other objects and advantages will be readily evident from the attached detailed description, drawings and claims to those skilled in the art.

### DESCRIPTION OF THE FIGURES

The present invention will be readily understood by the following detailed description in conjunction with the accompanying drawings, wherein like reference numerals designate like structural elements, and in which:

FIG. 1 is a diagrammatic view of a preferred embodiment of the present invention illustrating the logic controller console and an openable elbow or knee joint appliance with an internal circumferential array of coils, where the appliance and turns of the coils are openable by a multi-conductor buckle so as to be closable to complete the array of coils circuitry and close the appliance about the joint. (The coils are inside the appliance but are illustrated in solid lines for clarity.)

FIG. 2 is a simplified schematic diagram of the internal circuitry of the logic controller console of FIG. 1.

FIG. 3 is a perspective view of a knee or elbow, slide-on appliance of the invention with the internal array of coils illustrated in solid lines for clarity and a multi-conductor connector for connecting to the console of FIG. 1.

FIG. 4 is a front perspective view of a pelvic region appliance of the invention with its internal arrays of coils illustrated in solid lines for clarity and a multi-conductor connector for connecting to the console of FIG. 1.

FIG. 5 is a plan view of a simple strap appliance of the invention with its internal arrays of coils illustrated in solid lines for clarity and a multi-conductor connector for connecting to the console of FIG. 1.

FIG. 6 is a perspective view of a shoulder strap appliance with an internal array of coils in one strap illustrated in solid lines for clarity and a multi-conductor connector for connecting to the console of FIG. 1.

FIG. 7 is a variation of the FIG. 3 appliance, in a circumferential slide-on form for the elbow or knee joint, with an internal circumferential array of coils illustrated in solid lines for clarity and a multi-conductor connector for connecting to the console of FIG. 1.

FIG. 8 is a front perspective view of a buckle-up style pelvic appliance, with internal circumferential coils in each leg illustrated in solid lines for clarity, and multi-conductor buckles by which the appliance and the coils are closed about the pelvic region.

FIG. 9 is a plan view of a simple strap appliance of the invention with an internal circumferential coil laid open with its conductors illustrated here in solid lines for clarity, the conductors terminating in the multi-conductor edge components of the appliance buckle by which the appliance is closed about a region of the body to complete the coil circuit.

FIG. 10 is a perspective view of a shoulder strap appliance with internal circumferential arrays of coils in each cuff, the coils illustrated in solid lines here for clarity, with multi-conductor connectors for closing the coils of each cuff of the appliance and a multi-conductor connector for connecting the arrays of coils to the console of FIG. 1.

FIG. 11A is a perspective, partially laid open, view of the buckle form joint appliance of FIG. 8, with a close up view in FIG. 11B of the multi-conductor buckle connector by which the circuitry of the circumferential coil array, illustrated in solid lines for clarity, is completed when the appliance is closed.

FIG. 12 is a perspective view of a single strap coil embodiment of the invention, sufficiently long and flexible for multiple turns about a body part and configured with a multi-conductor buckle for completing the coil circuit.

FIG. 13 is a sectional view of a variation of the elbow or knee joint appliance illustrated in FIG. 1.

### DESCRIPTION OF THE PREFERRED EMBODIMENT

The present treatments for arthritis, musculoskeletal pain and muscular atrophy consist mostly of traditional medicine including physical therapy and pharmaceuticals with only small inroads made by advancing technology. One of the technologies that has been making significant progress in this field, with multiple scientific studies to support its efficacy, is pulsed electromagnetic stimulation (PES). To date, however, even this technology supported by the literature is not used extensively. This is due, in large part, to the expense associated with repeated clinic visits and trained healthcare operators required to use existing equipment.

The invention is susceptible of many and various embodiments; the preferred embodiments described below should not be interpreted as restrictive, but rather as merely illustrative of the invention.

Referring to the figures generally, the present invention obviates the need for continued healthcare provider or clinic-based intervention and allows the patient to administer therapy to the desired regions in an outpatient setting, after appropriate instruction and under the continuing supervision of a physician. The present invention includes an array of coils or a circumferential coil incorporated into a range of body appliances that use an insulated wrap design such that when the appliance is properly applied, and a current pulse is generated by the logic controller, an electromagnetic field is produced by the coils proximate the body area of interest. By placing the coils in an ergonomic appliance, clearly marked and labeled for correct positioning and repetitive placement, and providing the appropriate settings for the logic controller, the invention allows therapeutic PES to be utilized by patients in their homes without continued, costly healthcare provider demands.

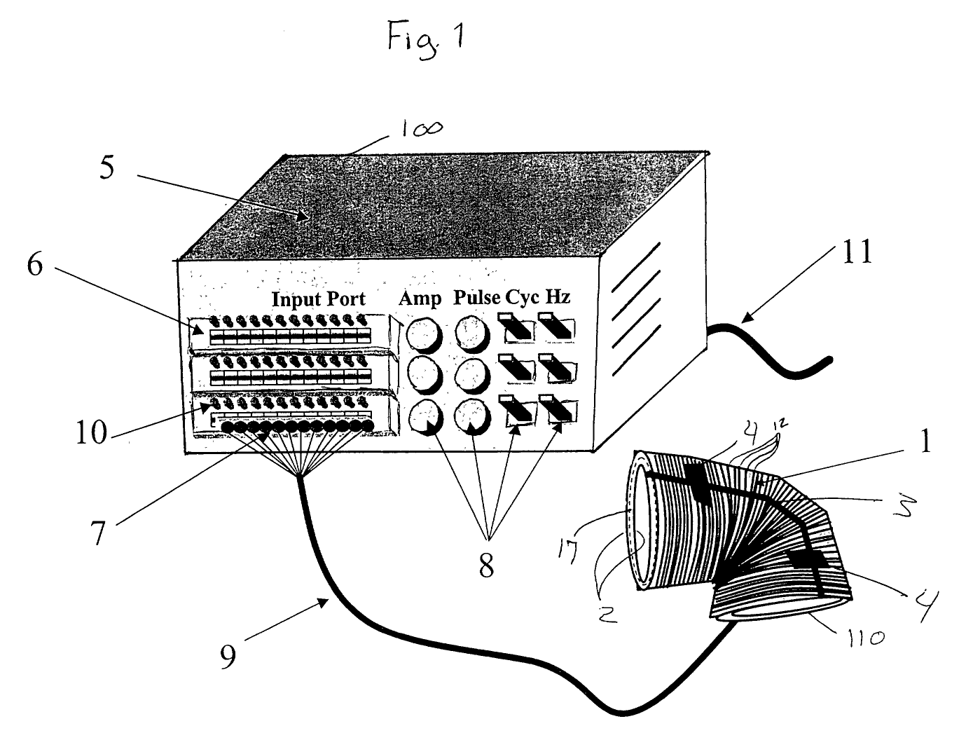

Referring to FIG. 1, there is displayed a logic controller console 100 attached by cable 9 to an elbow or knee joint appliance 110 which is configured with array of transducer coil 1 in accordance with the invention. Transducer coils 1 are insulated and placed between inner and outer electrically insulating layers 2, so as to achieve an orientation wherein the coils within the array are arranged coaxially with the appliance, and the treatment area with the electromagnetic field induced by an electric current in the coils approximately parallel to the treatment area. The joint appliance 110 has an openable, multi-conductor connector seam 3 common to all layers and to the transducer coils 1, allowing for laying open the appliance and array of coils for easy placement around the joint without having to be pulled on over the foot or hand. Closure of the connector seam 3 completes the circuitry of all coils 1 as well as closing the appliance about the joint. There is a pair or series of straps 4 allowing for securing of the placement of the appliance on the subject's joint and the connector seam 3. The straps may use snaps, buttons, hook and eye material or be replaced with zippers or other conventional fasteners or means for closing a wrap around garment. The circumferential and openable aspects and features of the array of coils 1 are further described below.

Adjacent to the transducer coils 1, between insulating layers 2, there is a conductive layer 17 of flexible wire mesh of the same or greater surface area than the array of coils 1, preferably the same surface area as the appliance overall, and disposed throughout the appliance so as to provide protection by grounding any defective coil and deactivating the power source at the logic controller (LC) console 100. The joint appliance 110 is connected to the logic controller console 100 through the multiple conductors of cable 9 and multi-conductor connector 7. The external LC console 100 consists of an inisulated shell 5, at least one connector port 6 for receiving the mating connector 7 from the appliance, adjustable controls 8 providing adjustment for the rate, amplitude, duration and number of pulses to be administered, individual port activation/deactivation selectors 10 to allow selection of which and in what order coils will be activated. Power cord 11 is connected to an ordinary a/c power source.

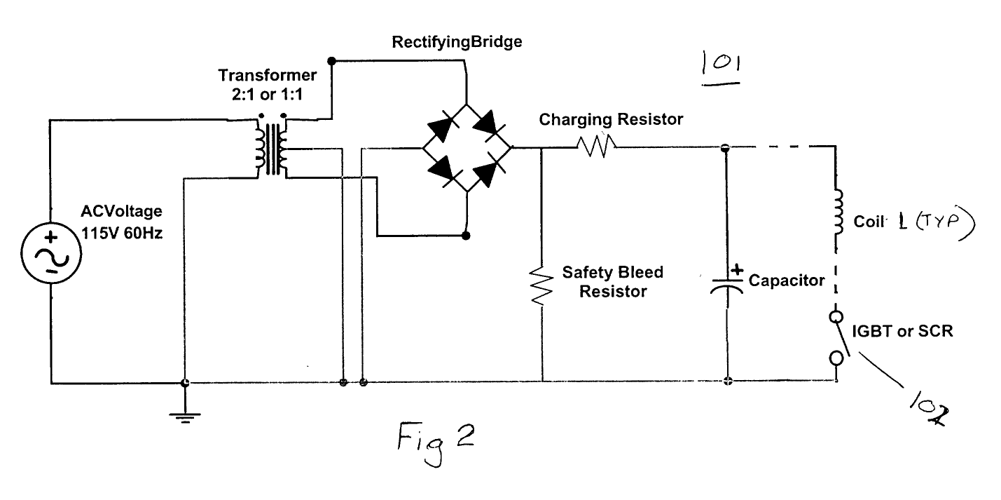

FIG. 2 illustrates the internal circuitry in simplified form of the logic controller 101 within console 100 of FIG. 1, with the basic components required to generate repetitive pulses. Coil switch 102, exemplary of a switch for each individual coil 1 of the selected appliance, can be triggered alone or in unison with other coil switches, providing the capability for stimulation of either a single or of multiple coils of the appliance, and allowing for varying pulse frequency and duration of each coil, and relative timing as between coils. Other and various embodiments of consoles and circuits suitable for use with the system as a whole will be readily apparent to those skilled in the art.

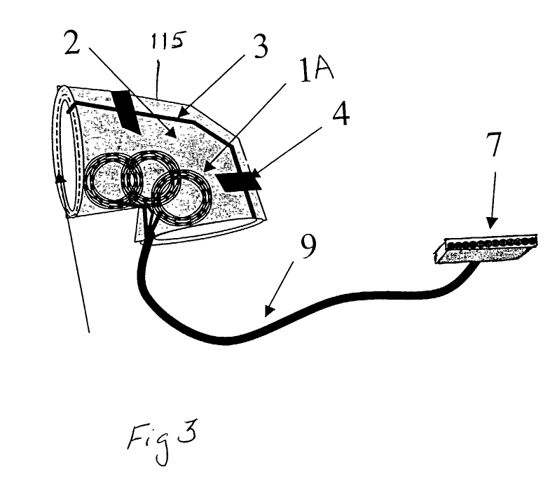

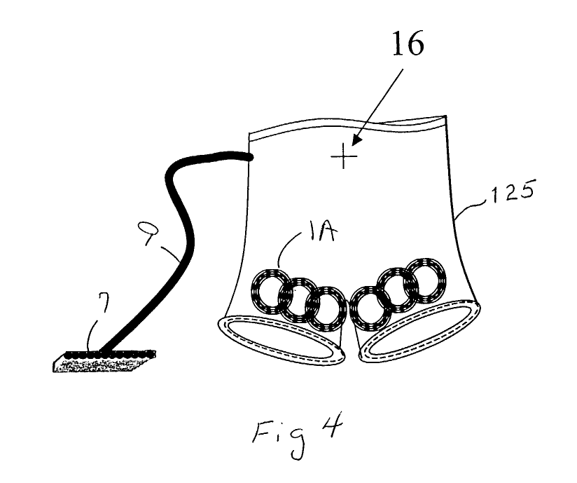

Referring to FIG. 3, there is illustrated an elbow or knee joint appliance 115, similar to the joint appliance 110 of FIG. 1, except that it is configured with an array of flat coils 1A, which when the appliance is secured properly to the joint, lie flat against the subject area, wherein the plane of the coils is substantially tangential to the surface of the target area. The coils of the array are intentionally displaced within the appliance to provide an extended area of coverage, and to permit manipulation of the pulse therapy by use of the control capabilities of console 100 to which the appliance is connectable by its connector 7.

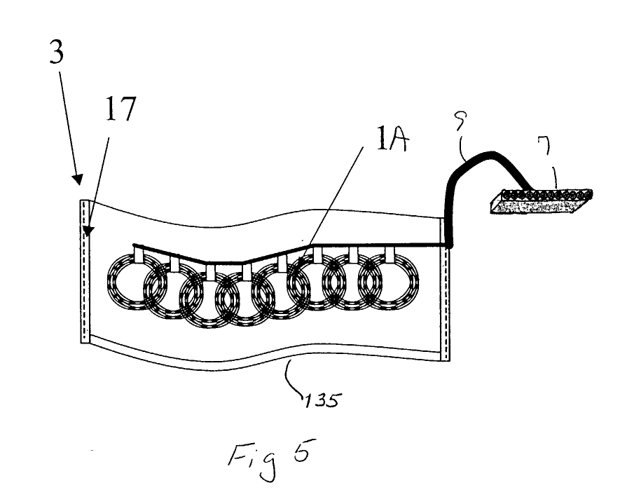

Referring now to FIG. 4, there is illustrated a pelvic region appliance 125 configured with internal arrays of coils, which when the appliance is properly worn by the patient, lie flat against the pelvic area. The coils, like those in the elbow or knee appliance of FIG. 3, are displaced to provide coverage of an extended area and manipulation of the pulse therapy by the use of the control capabilities of the console 100 to which the appliance is connectable by its connector 7. Also illustrated in FIG. 4 is one or more markings or indicia 16 that are designed to allow a prescribing physician to instruct an untrained patient how to accurately position the appliance, and to facilitate the patient's repetitive and consistent use of the appliance according to those instructions. Generally, the markings or indicia 16 are aligned with reference points on the patient's body, such a kneecap, vertebra, navel, or locus of pain, thereby insuring proper alignment of the appliance. While not illustrated in other figures, such reference markings or indicia 16 are equally well adapted to other embodiments of the appliance.

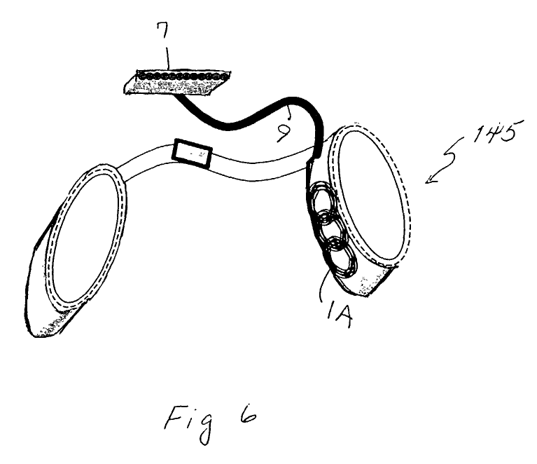

FIG. 5 illustrates a simple strap appliance 135 configured with internal arrays of coils, which when the appliance is worn by a patient, are designed to lie flat against the patient's body. The coils are displaced to allow coverage of an extended area and to permit manipulation of the electromagnetic field induced by the electric current flowing through the coils by the console 100 of FIG. 1 that is connected to the coils by a multi-conductor connector 7. The simple strap appliance 135 is provided for flexibility in providing treatments to areas for which an ergonomic appliance design has not been developed but for which the prescribing physician desires to employ PES therapy. Again, exterior markings or index symbols provide for repetitive placement of the appliance on the target region of the patient's body.

FIG. 6 illustrates another such appliance according to the present invention, a shoulder strap appliance for the stimulation of the shoulder joint, configured in such a way as to provide an array of internal coils that, when the appliance is secured properly to the joint, lie flat against the shoulder of the patient. The coils of the array, as in those embodiments illustrated in FIGS. 3-5, are intentionally displaced within the appliance to provide an extended area of coverage, and to permit manipulation of the pulse therapy by use of the control capabilities of console 100 to which the appliance is connectable by its connector 7.

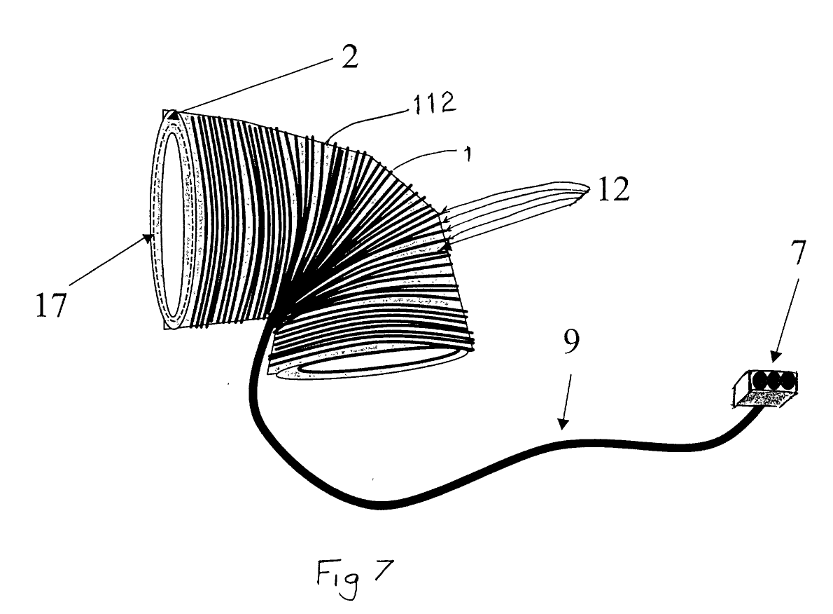

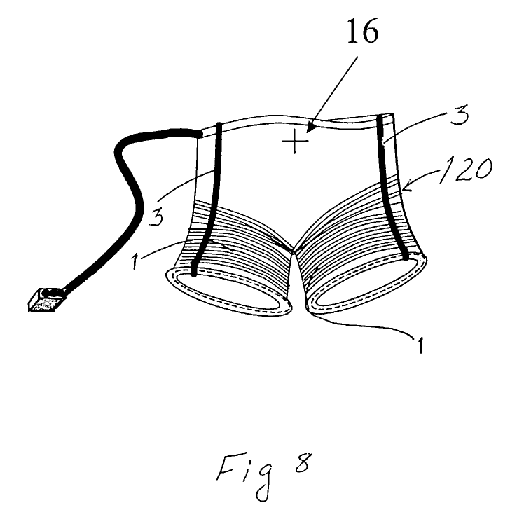

Referring now to FIG. 7, illustrating a circumferential slide-on form for the elbow or knee joint 112, a variation of the appliances 110 and 115 illustrated in FIGS. 1 and 3, respectively. In this embodiment, the appliance 112 is not openable, but has a sufficiently large diameter to permit the appliance to slip over the arm or leg of the patient, and into position. The array of coils, in this embodiment, is made up of at least one continuous wire that is wrapped many times around the circumference of the appliance, thus forming one or more solenoid or substantially solenoidal structure 1. The solenoid arrangement of the coil involves wrapping the continuous coil 1 around the treatment region. In all other respects this design is identical to the flat coil array of coils discussed above. As in the other embodiments, the wire is connected by a multi-conductor connector 7 to the console 100 of FIG. 1 and which controls and supplies an electric current that induces an electromagnetic field in the solenoid 1.

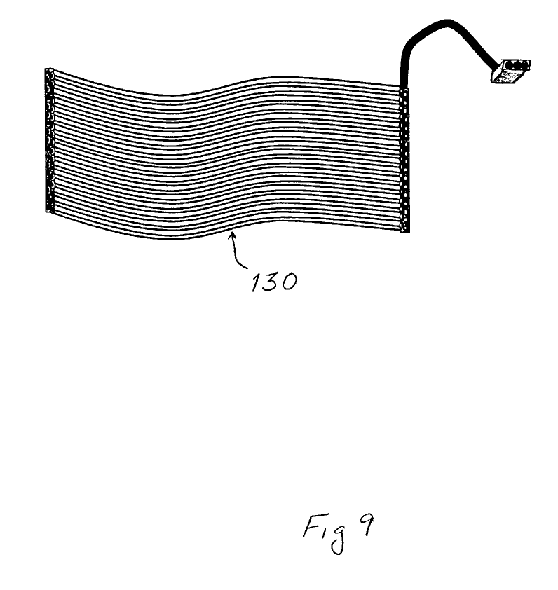

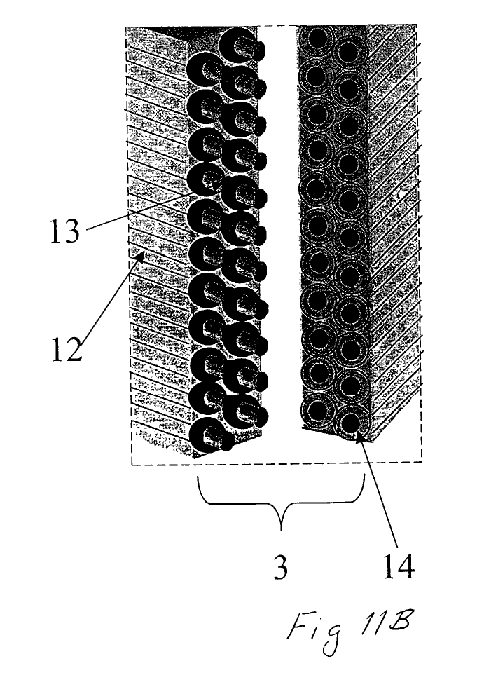

FIG. 8 illustrates a buckle-up style pelvic appliance configured with internal circumferential coils 1 in each leg. To enable the patient to easily put on and remove the appliance, this embodiment has multiple conductor connector joints 3 whereby the circumferential coils 12 may be interrupted and the appliance opened. Also illustrated in this embodiment are multi-conductor buckles by which the appliance and the coils are closed about the pelvic region. These elements are common to a number of embodiments 110,120,130, and 140. This system is illustrated in greater detail in FIGS. 11a and 11b.

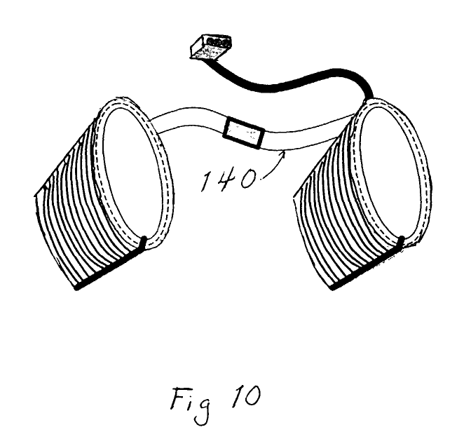

FIG. 9 illustrates a simple strap appliance 130 configured with an internal circumferential coil 1. The coil 1 is interrupted, with wire conductors 12 terminating in the multi-conductor edge components 13, 14 of the appliance 130 by which the appliance is buckled and closed about a region of the body to complete the coil circuit. When the appliance is closed, the wires are connected in such a way as to form a solenoid or substantially solenoidal structure 1. The strap appliance 130 is provided to allow physicians to prescribe PES therapy for areas of the body for which no ergonomic, or contour conforming appliance has yet been developed, or for which no such appliance is practical. As in all other appliances of the invention, strategically placed exterior markings provide for repetitive placement on the target area of the subject.

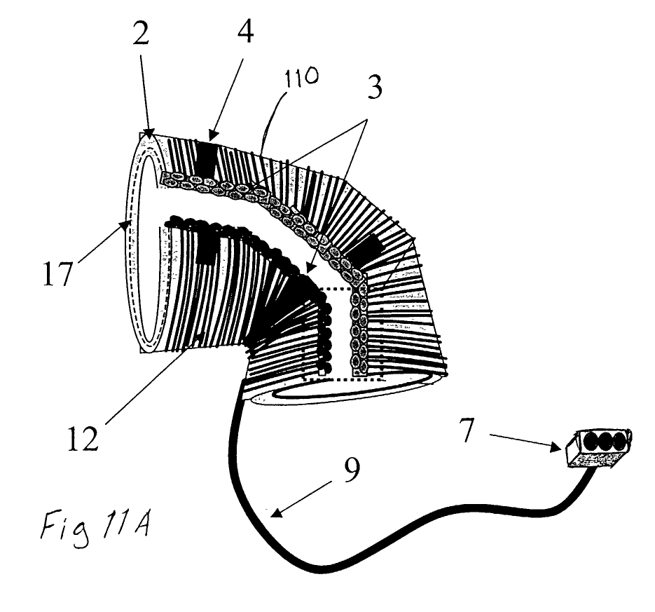

Referring now to FIG. 10 illustrates a shoulder strap appliance 140 with internal circumferential arrays of coils 1 in each cuff, with multi-conductor connectors 13,14 for closing the coils of each cuff of the appliance and a multi-conductor connector 7 for connecting the arrays of coils 1 to the console 100 of FIG. The configuration of the arrays of coils is substantially similar to that of the elbow or knee appliance 110, the pelvic area appliance 120, and the strap appliance 130.

FIG. 11A is a further illustration of the elbow or knee appliance 110, partially laid open to reveal the buckle form joint 3 of FIG. 1. FIG. 11B provides a detail view of the multi-conductor buckle connector by which the circuitry of the circumferential coil array 1 is completed when the appliance is closed. The joint 3 is composed of a plurality of paired male 13 and female 14 conducting pairings, disposed opposite to each other on opposite sides of the openable joint 3 of the appliance 110. Each conductive pair is insulated from its neighbors. A particular wire does not mate with itself. When closed the array of coils forms a substantially solenoidal structure.

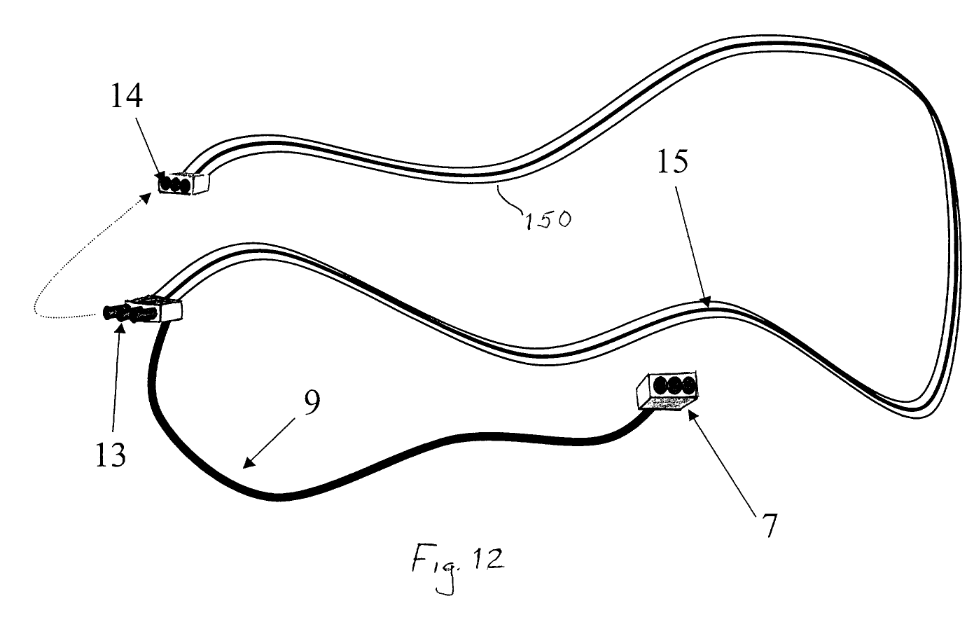

FIG. 12 illustrates yet another embodiment of the present invention, a single strap coil, this embodiment is substantially similar to the strap appliance 130, but comprises only a narrow register of wires, or even a single wire, those wires are sufficiently long and flexible to make multiple turns around a patient's body part and are configured with a multi-conductor buckle, as described above, for completing the coil circuit.

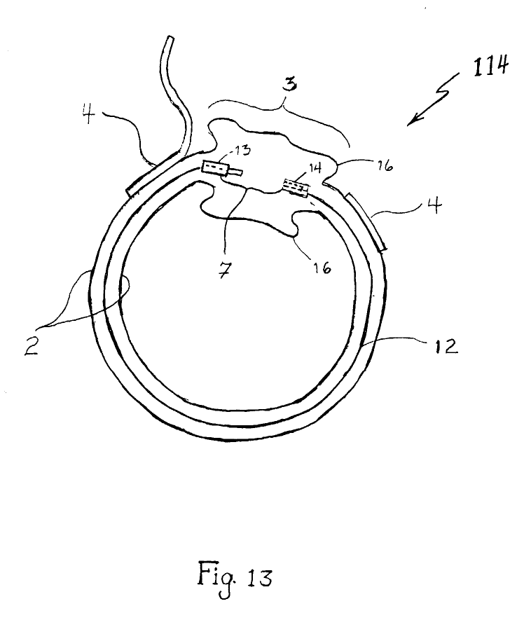

FIG. 13 is a sectional view of a variation of the elbow or knee joint appliance illustrated in FIG. 1. In this embodiment 114, the structure is substantially similar to that of the embodiment 110 in FIG. 1. The wires 12 terminate in male 13 and female 14 conducting pairings, which when joined link the wires, thus forming one or more solenoids. These wires and connectors are disposed between the insulating layers 2 of the appliance. In this embodiment 114, these insulating layers 2 have a greater circumference than the closed solenoid, so that when the connection 7 between the male 13 and female 14 conducting pairings is opened, the circumference of the appliance is substantially increased, allowing the patient to easily slide the appliance over the targeted limb. When the connection is made, the fabric of the extra circumference 16 is folded up, cinched, gathered, or gusseted by the solenoid and the straps 4.

One skilled in the art would readily see that the configurations of the coils disclosed herein and claimed below are designed to produce and channel an electromagnetic field through the tissues of a living organism in an efficient, repeatable, and simple way, and by way of use of the console and circuitry, customize patterns of pulsed electromagnetic fields, resulting in variable field form construction and time based application, which can be formulated to meet the requirements of the clinician.

Generally, in the embodiments discussed above, buckles or fasteners 4 may be provided to ensure secure attachment at the multiple conductor connector joint 3 and the logic controller will not send current pulses unless the buckles 4 are securely fastened.

Additional embodiments consistent with the invention designed but not shown in the drawings may include, but are not limited to appliances for the hand, foot, neck, head, ankle, face, abdomen, thorax, torso, full-body or half-body with coils placed to strategically stimulate desired tissues (ie the diaphragmatic nerve in the abdominal wrap for respiratory paralysis). Any of the tissues of the body may be stimulated according to this invention and subject to the constraints of nature. Examples of tissues include but are not limited to bone, muscle, organs, vascular tissue, arterial tissue, ligaments, tendons, skin and connective tissue.

The logic controller is a device through which electricity is provided to an array of coils. The logic controller controls the activation of the coils, as well as the rate of stimulation of a given coil. The logic controller also allows for the adjustment of stimulatory channels. One or more arrays of coils, preferably disposable, are plugged into the logic controller. The logic controller permits the activation or deactivation of individual coils, as well as adjustment of the rate of stimulation and thereby the amplitude of the stimulus, since higher frequency equates with less time for the capacitor to recharge and, therefore, a smaller discharge. Three or more arrays of coils may be plugged into the logic controller at once allowing for stimulation of multiple sites in sequence or together. The logic controller preferably weighs approximately 10 pounds. In other embodiments, the controller weighs less than 60 pounds; less than 40 pounds, less than 20 pounds, less than 5 pounds, or less than 2 pounds, depending on the condition being treated. The logic controller preferably plugs into a standard U.S. outlet (115 Volts, 10 Amps, 60 Hz). In alternative embodiments, the logic controller is designed to plug into a standard European outlet, a standard Japanese outlet, a standard Canadian outlet, or a standard Australian outlet. In other embodiments, the logic controller plugs into either an AC or a DC power source.

The internal circuitry of the logic controller comprises or alternatively consists of a transformer, a capacitor, a bridge rectifier, two resistors, an inducting coil, a diode, and a switch.

A transformer is a device that takes a first current and voltage and converts it to a second current and voltage through magnetic induction. The transformer (1:2) has an output of approximately 230 Volts and 5 Amps. In some embodiments, the transformer is 1:1 with an output of 115 volts (˜170 post bridge-rectifier). In alternative embodiments the transformer has an output of at least 50 volts, at least 150 volts, at least 250 volts, at least 450 volts, or at least 600 volts. In other alternative embodiments the transformer has an output of at least 1 Amp, at least 2.5 Amps, at least 5 Amps, at least 10 Amps, or at least 20 Amps. A bridge rectifier is an electrical component consisting of four diodes that when wired to a transformer will convert AC to DC.

The bridge rectifier converts the output voltage from AC to DC. The bridge rectifier must be rated to at least 1 kW and 350 Volts as the rectified output voltage will be approximately 320 Volts. In alternative 20 embodiments, the bridge rectifier is rated to at least 120 volts, at least 250 volts, at least 450 volts, or at least 600 volts. In other alternative embodiments, the bridge rectifier is rated to at least 100 W, at least 500 W, at least 2,000 W, or at least 5,000 W.

A capacitor and a stimulating coil are provided in parallel. The capacitor is at least 40,000 microfarads and is rated to 320 Volts, and provides approximately 2050 Joules/pulse. In alternative embodiments, the capacitor (or collection of capacitors in parallel) is at least 5,000 microfarads, at least 20,000 microfarads, at least 60,000 microfarads, at least 100,000 microfarads, at least 500,000 microfarads, at least 1,000,000 microfarads. In other alternative embodiments the capacitor is rated to at least 120 volts, at least 250 volts, at least 450 volts, or at least 600 volts. In yet another embodiment, the capacitor is rated to only 200 Volts at less than 7000 microfarads. In alternative embodiments, the capacitor is rated to no more than 120 volts, no more than 250 volts, no more than 450 volts, or no more than 600 volts, once again depending on the power requirement of the condition being treated. In other embodiments the capacitor is no more than 10,000 microfarads, no more than 9,000 microfarads, no more than 8,000 microfarads, no more than 6,000 microfarads, no more than 5,000 microfarads, or no more than 4,000 microfarads.

The inducting coil is any array of coils or solenoid that covers the treatment region for which the stimulation is intended. The array is preferably disposable, and is located external to the logic controller. One or more arrays may be attached to the logic controller as described in more detail below. Distal to the inductor is a switch that in one position allows the capacitor to discharge to ground through the stimulating coil. In the other position, the switch prevents the capacitor from discharging to ground through the stimulating coil. Each coil attached to the logic controller may have its own internal switching mechanism to allow firing of the coil in sequence or to allow the firing of multiple coils simultaneously.

In an embodiment, the switch activates a stimulation repetition rate of at least 1 Hz and a pulse duration of greater than 2 milliseconds. In other embodiments, the pulse duration is greater than 1 millisecond, greater than 5 milliseconds, greater than 25 milliseconds, greater than 50 milliseconds, greater than 100 milliseconds, greater than 150 milliseconds, or greater than 200 milliseconds. In other embodiments the stimulation repetition rate is at least 0.5, 15, 50, 100 Hz.

In an embodiment, where more than one coil exists, stimulation occurs through firing each coil individually, while using switches to short out the surrounding coils to prevent low-impedance interference. In alternative embodiments, additional coils are fired along with the target coil itself. The coil orientation will be such that the fields generated by the coils will be additive with maximum field generation being in the areas between the target and flanking coils. The choice of mode of stimulation will depend on the needs of the site of stimulation and will be programmable using methods and apparati well known to those skilled in the art.

In an embodiment, the step-up transformer is linked to the bridge rectifier, which is linked in parallel to a capacitor and inducting coil wherein the inducting coil is linked to a switch.

The array of coils may take a variety of configurations as long as the subcutaneous tissue is adequately carpeted. Designs in this invention include the flat coil array of coils, solenoid coil and the solenoid buckle coil. The coils may be disposable. In an embodiment, the coils overlay the tissue for which the stimulation is intended. The entire array of coils is enveloped in a non-conducting material (e.g. plastic, rubber, ceramic), over which is a thin layer of conducting mesh (aluminum, copper, steel, for example) and another layer of non-conducting material (e.g. plastic, rubber, ceramic) will be laid. Preferably, the layer of non-conducting material is a comfortable, but thin cloth allowing non-irritating application to the dermis. The material may be flocked, to be more comfortable. The conducting mesh will preferably be placed on both sides of the coils such that the logic controller will be disabled immediately if a short circuit occurs. If any current escapes the coil insulation, the conducting mesh will trap the current and blow an internal fuse disabling all current so as to protect the patient and/or caregiver. This safeguard helps to ensure that the patient and/or caregiver are not exposed to live wire.

In an embodiment, the coils are then incorporated into an ergonomic appliance, which is clearly marked and designed for optimal ease of application. These designs can include, but are not limited to, a shoulder harness, a pelvic harness, a knee applicator, an elbow applicator and a simple flat strap. The shoulder, pelvic, knee and elbow designs are preferentially ergonomically designed for ease of application and accuracy in targeting key nerves. Other possible designs include, but are not limited to, hand, foot, neck, face, full-body or half body (i.e. both legs) applicator wraps.

In one embodiment, each array will have coils strategically placed to treat the targeted tissues. This will not be the case with the simple strap application, which will have coils in a linear fashion throughout its entire length to make it a more flexible applicator.

The appliance may be fastened with Velcro. Other means of fastening include but are not limited to buckles, snaps, zippers, and hooks. Alternatively, the fabric is stretchable or expandable. The appliance may have a gusset between the multiple conductor connectors, thereby being expandable without opening completely. As used herein, an array is everything contained in one appliance. Appliances will be made in a variety of sizes to accept users of various sizes. The embodiments for the three types of wraps are further described below. In the flat coil design, coils are incorporated into the wrap perpendicular to the plane of the skin and multiple, independently stimulated coils are present throughout the wrap. This is in contrast to both of the solenoidal designs where the coil in the wrap consists of one, long continuous coil. In the preferred embodiment for the flat coil embodiment, each coil will be between 5 and 100 mm in diameter. In alternative embodiments, each coil will be at least 5 mm, at least 10 mm, at least 20 mm, at least 40 mm, at least 75 mm at least 100 mm, at least 125 mm, at least 150 mm, or at least 200 mm in diameter.

In one embodiment, each coil has a 40 mm diameter. In an embodiment each coil consists of insulated wire, either threaded or single core, for example, wrapped multiple times to obtain a toroidal configuration. The coils may be #12 insulated flat-wound copper wire wrapped 20 times. In other embodiments the coils are #10, #14, #16, #18, or #20 insulated flat-wound copper wire. In alternative embodiments, the wire is wrapped 5, 10, 20, 40, 75, 100, or 200 times, for example. Each coil will be its own insulated circuit connected to the logic 22 controller allowing the coils to be stimulated individually or in unison. In other embodiments, all the coils are activated in unison. The overlap between adjacent coils will be no more than 5%, 10%, 50%, or 75%. Various amounts of overlap will be tested for maximal field generation including the non-overlapping configuration. In preferred embodiments, the appliance itself is applied through an opening along one of its axes, that opening is then buckled. Alternatively, the wrap can be applied through drawing the wrap up over the treatment area if the treatment area makes this slide-on design possible. Otherwise the wrap will need to be opened and buckled.

In the solenoid coil design one long, continuous coil is incorporated into the appliance in such a manner that it surrounds the treatment region. Thus the embodiments vary greatly depending on the treatment region targeted. In embodiments of the solenoid coil, each coil will be of sufficient diameter to encompass the treatment region. Depending on the treatment region the diameter of the coil will vary widely from 3 cm to 50 cm with the only prerequisite being that the treatment area be completely enclosed. This range is wide due to the variety of treatment regions ranging from the wrist to the shoulder to the lower back. In an embodiment each coil consists of insulated wire, either threaded or single core, for example, wrapped multiple times to obtain a solenoid configuration. The coils may be #14 insulated flat-wound copper wire wrapped 50 times. In other embodiments the coils are #10, #12, #16, #18, #20 or #24 insulated, stranded or single core copper wire. In alternative embodiments, the wire is wrapped at least 20, 100, 300, or 500 times, for example. In some embodiments of the solenoid coil, the appliance is applied through drawing the appliance up over the treatment area. This appliance will only be useful in regions over which it can be comfortably slid on.

For all other regions, and for increased ease of application, the solenoid buckle coil design will be required. In this design, similar to the solenoid coil, one long, continuous coil is incorporated into the wrap in such a manner that it surrounds the treatment region. The main difference between the solenoid coil and the solenoid buckle coil lies in the method of application. For the solenoid buckle coil, the stimulatory coil is interrupted throughout its length on one of its sides so that the application can be completely opened to allow the treatment region to be comfortably and easily placed inside. Once the treatment region is inside the application, the opening is closed and buckled such that the wire interfaces are reconnected creating one long continuous coil electrically indistinguishable from a solenoid coil. In embodiments of the solenoid buckle coil, each coil will be of sufficient diameter to encompass the treatment region. Depending on the treatment region the diameter of the coil will vary widely from 3 cm to 50 cm with the only prerequisite being that the treatment area be completely enclosed. This range is wide due to the variety of treatment regions ranging from the wrist to the shoulder to the lower back. In the preferred embodiment each coil consists of insulated wire, either threaded or single core, for example, wrapped multiple times to obtain a solenoid configuration. The coils may be #14 insulated flat-wound copper wire wrapped 50 times. In other embodiments the coils are #10, #12, #16, #18, #20 or #24 insulated, stranded or single core copper wire. In alternative embodiments, the wire is wrapped at least 20, 100, 300, or 500 times, for example.

In one embodiment, the application consists of an interface where the wire in the application is interrupted and a male and female adapter are attached, one on each side. Once the treatment region is inside the opened application, the male and female adapters are joined creating one long continuous coil. As a safety feature, these adapters can only be attached in one configuration and the opening must be securely buckled prior to allowing any current impulse conduction. In one embodiment, the solenoid coil application is applied through opening the interface and inserting the treatment region after which the interface is securely buckled.

The present invention is intended for use in any condition in which the treatment or prevention of muscular atrophy or augmentation of muscle mass, the treatment of musculoskeletal pain or the treatment of arthritis is desired.

Conditions in which this invention would be indicated for prevention or treatment of atrophy include, but are not limited to: muscle wasting syndromes such as cancer, AIDS, and myositis, and conditions resulting in immobilization or disuse atrophy such as paralysis, Guillain-Barre, routine fractures, coma, and sedentary or bedridden patients. Furthermore, the present invention could be used as a means of muscular augmentation outside of the medical field by athletes and those desiring increased muscle mass. Alternatively, the invention is useful for the prevention of deep venous thrombosis in immobilized patients through stimulation of the musculature in their lower extremities. In addition, the device could be used in the treatment of non-healing wounds/fractures.

Conditions in which this invention would be indicated for musculoskeletal pain include, but are not limited to: chronic or acute back pain, chronic or acute neck pain, tendonitis, bursitis, bone pain or any other chronic or intractable pain. Conditions in which this invention would be indicated for arthritis include all stages of osteoarthritis, psoriatic arthritis and rheumatoid arthritis.

For therapeutic use, the invention will require different settings for use based on the condition to be treated and the embodiment to be used. For treatment of muscular atrophy, for example, large current pulses will be required along with the flat coil or small diameter solenoid coil. This treatment regimen will generate magnetic fields in excess of 2 Tesla (20,000 gauss) whereas the treatment of arthritis and musculoskeletal pain will require a much weaker magnetic field in the range of 10-500 gauss.

Furthermore the different indications will also require different frequencies of treatment and different current pulse settings. The potential methods of use for the three conditions are given as follows. For muscular atrophy, current pulses should be of sufficient power to stimulate the muscle and should be used at least twenty minutes each day for each site of stimulation in two ten minute increments spaced 4-8 hours apart. This is a minimum and these values may be increased as the care provider sees fit for accelerated recovery. Alternative treatments include 15 minutes treatments spaced 20 minutes apart and all increments up to and including 10 minute treatments spaced 24 hours apart. Therapy should last at least a week with the upper end being months (or even years) for patients with conditions resulting in long-term immobilization. The stimulation should result in contraction of the musculature, preferably near supramaximal values assessed through observation and physical exam. The actual contraction strength, though, may be therapeutic with only induction of twitches and not require supramaximal contractions. The applicators should be placed in their intended positions, (i.e. elbow coils on the elbow, not the knee) due to the strategic placement of stimulatory coils in each unit. The applicators are designed such that they fit ergonomically and require little thought or effort in placing them.

For musculoskeletal pain this application will typically, but not exclusively, employ the solenoid coil and solenoid buckle coil designs. These coils will receive current pulses raging from 1 Amp to 100 Amps at frequencies between 1 Hz and 75 Hz to provide the required analgesic effect without stimulating nerve or muscle. The treatment regimen will require the patient to use the device every 4-6 hours as needed for pain with each treatment session lasting anywhere between 10 and 30 minutes. Alternatively, the device may be used anywhere from a continuous basis (as a portable device with a portable LC) to an as needed basis which may entail therapy once every week or even less. The applicators should be placed in their intended positions, (i.e. elbow coils on the elbow, not the knee) due to the strategic placement of the coil in each unit. The applicators are designed such that they fit ergonomically and require little thought or effort in placing them.

As with the musculoskeletal pain indication, the treatment of arthritis will typically, but not exclusively, employ the solenoid coil and solenoid buckle coil embodiments. These coils will receive current pulses raging from 1 Amp to 100 Amps at frequencies between 1 Hz and 75 Hz to the therapeutic effect without stimulating nerve or muscle. The treatment regimen will require the patient to use the device twice a day for duration of 30 minutes. After sufficient treatment duration, the patient will notice decreased pain and increased range of motion after which the treatment frequency may be reduced.

Alternatively, the device may be used anywhere from a continuous basis (as a portable device with a portable LC) to an as needed basis which may entail therapy once every week or even less. The applicators should be placed in their intended positions, (i.e. elbow coils on the elbow, not the knee) due to the strategic placement of the coil in each unit. The applicators are designed such that they fit ergonomically and require little thought or effort in placing them.

A possible embodiment of the present invention is a system for the electromagnetic stimulation of living tissue including a flexible and conformable appliance having first and second electrically insulating layers, between which are disposed at least one coil of insulated conductive material, through which runs an electric current, controlled by a controller, thereby inducing pulsed electromagnetic fields. The first electrically insulating layer is adapted to be in contact with a targeted part of a patient's body and the second electrically insulating layer has at least one clearly visible marking disposed so as to facilitate repeatable alignment of the appliance with the targeted part of the patient's body. In this embodiment an appliance is configured to ergonomically fit the target part of the patient's body.

In this the plane of said coil may be arranged tangentially to a surface of said targeted part of said patient's body. The appliance may include an openable joint disposed in the appliance. The controller may independently control each coil. There may be a plurality of coils arranged in an array. The coils in said array may be arranged to allow the pulsed electromagnetic fields to overlap or coils in said array may be arranged to overlap.

The appliance, of this embodiment, may also be configured to be coaxial with the targeted part of the patient's body. The coils may be arranged coaxially with and disposed circumferentially within said appliance. First and second multi-conductor connectors may connectably interrupt the coils. The appliance may have an openable joint allowing the appliance to be comfortably applied to the part of the patient's body. Alternatively the appliance may be comprised of expandable material. The appliance may be configured to be expandable. This appliance may also include a conductive layer disposed between the coils and the first and second insulating layers, whereby the controller is triggered to interrupt the electric current in the event of a failure in insulation of the insulated conductive material.

Another embodiment is a system for the electromagnetic stimulation of living tissue including an appliance having interior and exterior coverings, the appliance may be ergonomically designed, flexible, and electrically insulated. A plurality of wires may be disposed between the interior and exterior coverings, each wire having first and second ends, these first and second ends are connected to first and second conductive interface structures respectively, whereby, when the first and second interface structures are joined together, the wires are connected in such a way as to form at least one transducer, possibly, a solenoid. This embodiment may also include a control apparatus, controlling an electrical current flowing through the transducer, thereby inducing a pulsed electromagnetic field.

The appliance may have a central diameter wide enough to surround at least part of a body through the solenoid, which may be substantially coaxial with said appliance. The appliance may be adapted to comply with the contours of at least part of a living organism, whereby the electromagnetic field may be induced within the tissues of that organism. The appliance may be expandable thereby allowing the appliance to be slipped over a part of the body. To achieve this, the appliance may be gusseted or have an openable joint.

Marking indicia may be placed on the appliance to enable a user to position the appliance such that the transducers are properly disposed to enable optimum nerve stimulation. Markings may also be placed on the appliance in such a way as to facilitate repeatable alignment of said appliance with said part of said body.

Another possible embodiment of the present invention is an apparatus for the electromagnetic stimulation of living tissue that is an appliance, wherein that appliance is ergonomically designed, flexible, and electrically insulated, has at least one clearly visible marking disposed on the appliance so as to facilitate repeatable alignment of the appliance with at least a targeted part of a patient's body. The appliance also may have a plurality of wires disposed within the appliance, each wire having first and second ends, these first and second ends may be connected to first and second conductive interface structures respectively, whereby, when these first and second interface structures are joined together, the wires are connected in such a way as to form at least one transducer, such as a solenoid, connected by a communications tether to an external logic control console which controls an electric current flowing through the transducer, wherein the current induces a pulsed electromagnetic field. The transducer may be disposed coaxially with and substantially integrally in the appliance, that has a central diameter wide enough to allow the introduction of at least the targeted part of the patient's body through the solenoid. The said appliance may be adapted to comply with the contours of at least part of a human being, whereby an electromagnetic field may be induced within the tissues of the human being.

The appliance may be expandable, gusseted or have an openable joint. The first and second conductive interface structures together may comprise a conductor connecting buckle. In these embodiments, the appliance may disposable.

The objects and advantages of the present invention may be further realized and attained by means of the instrumentalities and combinations particularly pointed out in the appended claims. Accordingly, the attached drawings and the above description are to be regarded as illustrative in nature, and not as restrictive.

Although specific features of the invention are shown in some drawings and not in others, this is for convenience only as each feature may be combined with any or all of the other features in accordance with the invention. The words “including”, “comprising”, “having”, and “with” as used herein are to be interpreted broadly and comprehensively and are not limited to any physical interconnection. Moreover, any embodiments disclosed in the subject application are not to be taken as the only possible embodiments.

I claim:

1. A system for the electromagnetic stimulation of living tissue comprising:
a flexible and conformable appliance comprising first and second electrically insulating layers, between which are disposed
at least one coil of insulated conductive material, through which runs an electric current, controlled by
a controller, thereby inducing pulsed electromagnetic fields;
wherein said first electrically insulating layer is adapted to be in contact with a targeted part of a patient's body and said second electrically insulating layer has at least one clearly visible marking disposed so as to facilitate repeatable alignment of said appliance with said targeted part of said patient's body; and
wherein said appliance is configurable to ergonomically fit said target part of said patient's body.

2. The system for electromagnetic stimulation of living tissue of claim 1 wherein the plane of said coil is configured within said appliance for placement tangential to a surface of said targeted part of said patient's body.

3. The system of electromagnetic stimulation of living tissues of claim 2 further comprising an openable joint disposed in said appliance.

4. The system for electromagnetic stimulation of living tissue of claim 2 wherein said controller independently controls each said coil.

5. The system for electromagnetic stimulation of living tissue of claim 2 wherein said at least one coil is a plurality of coils arranged in an array.

6. The system for electromagnetic stimulation of living tissue of claim 5 wherein said plurality of coils in said array is arranged to allow said pulsed electromagnetic fields to overlap.

7. The system for electromagnetic stimulation of living tissue of claim 5 wherein said coils in said array are overlaping.

8. The system for electromagnetic stimulation of living tissue of claim 1 wherein said appliance is configured to be coaxial with said targeted part of said patient's body.

9. The system for electromagnetic stimulation of living tissue of claim 8 wherein said coils are arranged coaxially with said appliance, and with conductors disposed circumferentially within said appliance.

10. The system of electromagnetic stimulation of living tissues of claim 9 wherein first and second multi-conductor connectors connectably interrupt said coils.

11. The system of electromagnetic stimulation of living tissues of claim 10 wherein said appliance further comprises an openable joint whereby allowing said appliance to be comfortably applied to said part of said patient's body.

12. The system of electromagnetic stimulation of living tissues of claim 10 wherein said appliance is comprised of expandable material.

13. The system of electromagnetic stimulation of living tissues of claim 10 wherein said appliance is configured to be expandable.

14. The system of electromagnetic stimulation of living tissues of claim 1 further comprising a conductive layer disposed between said coils and said first and second insulating layers, whereby said controller is triggered to interrupt said electric current in the event of a failure in insulation of said insulated conductive material.

15. A system for the electromagnetic stimulation of living tissue comprising:
an appliance having interior and exterior coverings, wherein said appliance is ergonomically designed, flexible, and electrically insulated, in which are disposed
a plurality of wires disposed between said interior and exterior coverings, each wire having first and second ends, said first and second ends are connected to
first and second conductive interface structures respectively, whereby, when said first and second interface structures are joined together, said wires are connected in such a way as to form at least one
transducer, wherein said transducer is a solenoid,
a control apparatus, controlling an electrical current flowing through said transducer, thereby inducing a pulsed electromagnetic field,
wherein said appliance has a central diameter wide enough to surround at least part of a body through said solenoid,
wherein said solenoid is substantially coaxial with said appliance, and
wherein said appliance is adapted to comply with the contours of at least part of a living organism, whereby said electromagnetic field may be induced within the tissues of said organism.

16. The system for the electromagnetic stimulation of living tissue of claim 15, wherein said appliance is expandable thereby allowing the appliance to be slipped over said part of said body.

17. The system for the electromagnetic stimulation of living tissue of claim 16, wherein said appliance is gusseted.

18. The system for the electromagnetic stimulation of living tissue of claim 15, wherein said appliance has an openable joint.

19. The system for the electromagnetic stimulation of living tissue of claim 15, further comprising marking indicia disposed on said appliance to enable a user to position said appliance such that said transducers are properly disposed to enable optimum nerve stimulation.

20. The system for the electromagnetic stimulation of living tissue of claim 15 wherein said appliance has at least one marking disposed on it in such a way as to facilitate repeatable alignment of said appliance with said part of said body.

21. An apparatus for the electromagnetic stimulation of living tissue comprising:
an appliance, wherein said appliance is ergonomically designed, flexible, and electrically insulated,
at least one clearly visible marking disposed on said appliance so as to facilitate repeatable alignment of said appliance with at least a targeted part of a patient's body
a plurality of wires disposed within said appliance, each wire having first and second ends, said first and second ends are connected to
first and second conductive interface structures respectively, whereby, when said first and second interface structures are joined together, said wires are connected in such a way as to form at least one
transducer, wherein said transducer is a solenoid, connected to
a communications tether, connected to, and whereby
an external logic control console controls an electric current flowing through said transducers, wherein said current induces a pulsed electromagnetic field,
wherein said transducer is disposed coaxially with and substantially integrally in said appliance,
wherein said appliance has a central diameter wide enough to allow the introduction of at least said targeted part of said patient's body through said solenoid,
wherein said appliance is adapted to comply with the contours of at least part of a human being, whereby an electromagnetic field may be induced within the tissues of said human being.

22. The electromagnetic stimulating device of claim 21 wherein said appliance is expandable.

23. The electromagnetic stimulating device of claim 21 wherein said appliance is gusseted.

24. The electromagnetic stimulating device of claim 21 wherein said appliance has an openable joint.

25. The electromagnetic stimulating device of claim 21 wherein said first and second conductive interface structures together comprise a conductor connecting buckle.

26. The electromagnetic stimulating device of claim 21, wherein said appliance is disposable.
